(19) United States
(12) Patent Application Publication
(10) Pub. No.: US 2020/0000643 A1
(43) Pub. Date: Jan. 2, 2020

(54) Long-Duration, Deep Wound Filler With Means To Prevent Granulation In-Growth

(71) Applicant: KCI Licensing, Inc., San Antonio, TX (US)

(72) Inventor: Christopher Brian LOCKE, Bournemouth (GB)

(21) Appl. No.: 16/418,721

(22) Filed: May 21, 2019

Related U.S. Application Data
(60) Provisional application No. 62/691,484, filed on Jun. 28, 2018.

(51) Int. Cl. A61F 13/02; A61F 13/15; A61F 13/00

(52) U.S. Cl. CPC A61F 13/0216; A61F 13/15577; A61F 2013/00357; A61F 2013/00093; A61F 13/00063

(57) ABSTRACT

Systems, apparatuses, and methods for providing negative pressure to a tissue site are disclosed. Illustrative embodiments may include an apparatus or system comprising a dressing for treating a tissue site with negative pressure. For example, the dressing may comprise or consist essentially of a manifold and a contact layer. In some embodiments, the manifold may have a tubular shape with a central axis. The contact layer may comprise a polymer film completely or substantially enclosing the manifold in some examples. In further embodiments, the dressing may comprise a plurality of fluid restrictions in the contact layer, the fluid restrictions configured to open or expand in response to a pressure gradient across the polymer film.

### RELATED APPLICATION

The present invention claims the benefit, under 35 U.S.C. § 119(e), of the filing of U.S. Provisional Patent Application No. 62/691,484, filed Jun. 28, 2018, which is incorporated herein by reference for all purposes.

### TECHNICAL FIELD

The invention set forth in the appended claims relates generally to tissue treatment systems and more particularly, but without limitation, to dressings for tissue treatment and methods of using the dressings for tissue treatment.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound can be washed out with a stream of liquid solution, or a cavity can be washed out using a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

For example, in some embodiments, a dressing for treating a tissue site, such as a deep or tunnel wound, may comprise a manifold having a tubular shape. The manifold may be foam in some embodiments. For example, a hydrophilic, felted foam may be particularly advantageous for some applications. A contact layer having a plurality of fluid restrictions may substantially enclose the manifold. In some examples, the contact layer may comprise or consist essentially of a polymer film, such as a film of polyurethane, polyethylene, silicone, or other material having suitable flexibility and bio-compatibility properties. The contact layer preferably has little or no surface texture, and may also be highly hydrophobic in some examples. In some embodiments, the perforations may be slits or slots.

In some examples, the contact layer may wrap around and be bonded to the manifold, may be sprayed to the manifold, may be formed by a secondary heat-sealing process, or may be flame-laminated to the manifold. In some examples, the contact layer may be welded to form a tube, and the manifold may be placed within and welded to the tube.

The fluid restrictions may be configured to remain open under a therapeutic pressure. In some examples, the fluid restrictions may be perforations in the contact layer, and may be oriented at about 45 degrees to horizontal.

In some embodiments, the manifold may additionally have a longitudinal perforation down a central axis of the manifold. The perforation may have a diameter of between 2 millimeters and about 5 millimeters prior to being covered by the contact layer. The perforation may be directly connected to a source of negative pressure, instillation solution, or other fluid in some examples. Additionally, a perforated polymer structure may be disposed within the central perforation to facilitate channeling fluids through the length of the manifold.

More generally, some embodiments of a dressing for treating a tissue site with negative pressure may comprise or consist essentially of a manifold and a polymer film. In some embodiments, the dressing may comprise a manifold having a tubular shape with a central axis. The dressing may comprise a polymer film completely or substantially enclosing the manifold in some examples. In further embodiments, the dressing may comprise a plurality of fluid restrictions in the polymer film, the fluid restrictions configured to open or expand in response to a pressure gradient across the polymer film.

In more specific examples, the polymer film may be hydrophobic. In some further examples, the polymer film may have a contact angle with water greater than 90 degrees. Examples of suitable polymer films may include, without limitation, polythene, polyurethane, acrylics, polyolefines, polyacetates, polyamides, polyesters, polyether block amide, thermoplastic vulcanizates, polyethers, and polyvinyl alcohol. In further embodiments, the polymer film is a polyethylene film. In some embodiments, an area density of the polymer film of less than 40 grams per square meter may be suitable, and an area density of less than 30 grams per square meter may be particularly advantageous for some applications.

In some embodiments, the fluid restrictions in the polymer film of the dressing may comprise a plurality of slots configured to permit fluid flow and inhibit exposure of the manifold to the tissue site, such as a deep wound or tunnel wound. In further embodiments, each of the plurality of slots may have a line of symmetry that forms an oblique angle with the central axis of the manifold. Particularly, the oblique angle may be in a range of about 30 degrees to about 60 degrees, such as about 45 degrees.

The fluid restrictions may comprise or consist essentially of elastomeric valves in the polymer films that are normally closed. For example, the elastic passages are responsive to a pressure gradient. For example, the fluid restrictions may comprise or consist essentially of fenestrations, slits, or slots in the polymer film that open or expand in response to a pressure gradient. In some embodiments, the fluid restrictions in the polymer film may comprise a plurality of fenestrations, slits, or slots that form an oblique angle with the central axis of the manifold or a longitudinal axis of the polymer film. For example, the oblique angle is in a range of about 30 degrees to about 60 degrees, or more particularly, about 45 degrees.

In some examples, the fluid restrictions may comprise or consist of a plurality of slits or slots in the polymer film. One or more of the plurality of slits or slots may have a length of at least 2 millimeters and not greater than 4 millimeters. In particular examples, each of the plurality of slits or slots may have a length of at least 2 millimeters and not greater than 4 millimeters, or more particularly, a length of about 3 millimeters. The fluid restrictions may comprise or consist of a plurality of slits or slots having a width of at least 0.5 millimeters and not greater than 2 millimeters. In particular examples, each of the plurality of slits or slots may have a width of at least 0.5 millimeters and not greater than 2 millimeters. Slits or slots with a length of at least 2 millimeters and not greater than 4 millimeters and a width of at least 0.5 millimeters and not greater than 2 millimeters may be particularly suitable for many applications.

Slits or slots of such configurations may function as imperfect valves that substantially reduce liquid flow in a normally closed or resting state. For example, such slits or slots may form a flow restriction without being completely closed or sealed. The slits or slots may remain open, expand, or open wider in response to a pressure gradient to allow increased liquid flow.

In some embodiments, the manifold may be hydrophilic. For example, the manifold may comprise a foam. In more particular examples, the manifold may comprise or consist essentially of a polymer foam, or more particularly, a polyurethane ester foam. The manifold may comprise or consist essentially of an open-cell foam, a reticulated foam, a reticulated polymer foam, a reticulated polyurethane ester foam, a felted foam, a non-felted foam, or any suitable foam or polymer.

In some embodiments, a dressing may comprise a manifold with a tubular shape having a circular cross-section or a semi-circular cross-section. In other embodiments, the tubular shape may have an elliptical cross-section or a polygon cross-section. In other further embodiments, the tubular shape may have a cross-section of a square, a hexagonal or any available shape. The tubular shape may have a solid core in some examples. In other examples, the tubular shape may have a hollow core.

In further embodiments, a dressing may comprise a plurality of bonds between portions of the polymer film, wherein the bonds are configured to define separable sections of the manifold, the polymer film, or a combination thereof. The plurality of bonds may define the separable sections or may form seams between the separable sections, for example, at an interval of about 3 centimeters to about 5 centimeters. In some embodiments, the separable sections or seams may have a length in a range of about 5 millimeters to about 8 millimeters. The dressing may further comprise perforations through the manifold alight with the bonds between portions of the polymer film. In additional embodiments, the manifold may comprise perforations through the manifold aligned with the bonds between the separable portions of the polymer film.

In some embodiments, a dressing for treating a tissue site with negative pressure may comprise a manifold comprising a first surface and a second surface opposite the first surface; an envelope around the manifold, the envelope defined by a first polymer film and a second polymer film coupled to a periphery of the first polymer film; and a plurality of fluid restrictions in the envelope, the fluid restrictions configured to remain open or expand in response to a pressure gradient across the polymer film. In further embodiments, the manifold may be formed into a tubular shape, and the envelope may comprise a first edge and a second edge coupled to the first edge to retain the tubular shape.

An apparatus for treating a tissue site with negative pressure is also described herein, wherein some example embodiments include a tissue interface comprising a manifold and a film substantially or completely enclosing, enveloping, or surrounding the manifold; a plurality of fluid restrictions in or through the film, the plurality of fluid restrictions configured to open or expand in response to a pressure gradient across the film. The tissue interface may further comprise a sealing layer in some embodiments, which may be disposed adjacent to the film and configured to contact the tissue site. Some embodiments of the apparatus may additionally include a negative-pressure source, a fluid source, or a combination thereof, fluidly coupled to the tissue interface.

In some examples, a method of preventing granulation in-growth in a deep or tunnel wound may comprise applying a dressing to the wound, wherein the dressing comprises a manifold and a film substantially or completely enclosing, enveloping, or surrounding the manifold. The film may comprise a plurality of fluid restrictions configured to remain open or expand in the presence of a pressure gradient, particularly, a negative pressure therapy, across the film. The film and the manifold may be described as above, for example, the manifold may be surrounded by a film with fluid restrictions that form an oblique angle with a central axis or a longitudinal axis of the manifold or the film. The dressing may be fluidly coupled to a negative-pressure source, and negative pressure from the negative-pressure source may be applied to the dressing.

Non-limiting advantages of the claimed subject matter may include reduced risk of granulation in-growth and infection during treatment, which may enable an extended wear of an improved dressing (for example, a change frequency of more than four days), increased therapy compliance, and decreased costs of care. Other objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide tissue treatment in accordance with example embodiments of this specification;

FIG. 2 is a perspective view of an example configuration of a tissue interface that may be associated with some example embodiments of the therapy system of FIG. 1.

FIG. 3 is a partial side cut-away view of the tissue interface of FIG. 2, illustrating additional details that may be associated with some example embodiments of the therapy system of FIG. 1.

FIG. 4 is a partial detailed view of the tissue interface of FIG. 3.

FIG. 5 is a top view of another example configuration of a tissue interface, illustrating additional details that may be associated with some embodiments.

FIG. 6 is a section view of the tissue interface of FIG. 5, illustrating additional details that may be associated with some examples.

FIGS. 7A-7C illustrate another example configuration of a tissue interface in various stages of assembly.

### DESCRIPTION OF EXAMPLE EMBODIMENTS

The following description of example embodiments provides information that enables a person skilled in the art to make and use the subject matter set forth in the appended claims, and may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

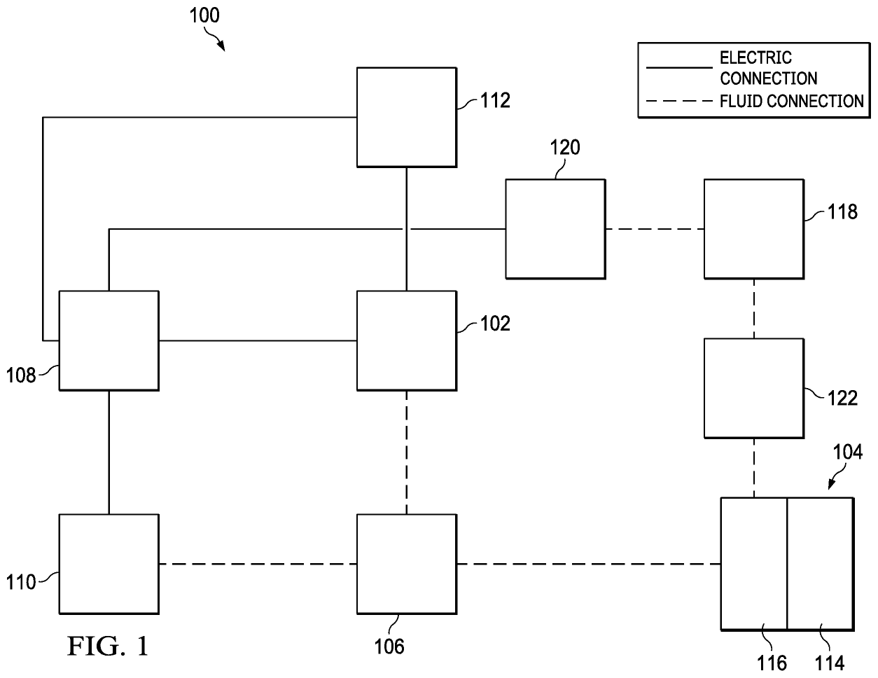

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including but not limited to, a tunnel wound site, such as a puncture or a fistula, a surface wound, a post-operative incision, a compartmented tissue, a compartmented wound site, an overhand wound, a bone tissue, an adipose tissue, a muscle tissue, a neural tissue, a dermal tissue, a vascular tissue, a connective tissue, a cartilage, tendons, or ligaments. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

In some embodiments, the tissue site may be a tunnel wound. As used herein, the term “tunnel wound” may broadly refer to a wound or defect that has an opening or passageway underneath the skin and tunnels into a patient's soft tissue. A tunnel wound may result in dead space with potential for abscess formation. For example, a tunnel wound may have a proximal opening, which may or may not be on a wound bed, and has a bottom at a distal end. A tunnel wound may extend in any direction through soft tissue underneath the skin. Tunnel wounds may pose complication risk that is due to the difficulty in removing exudate or other fluids from the tunnel wound.

In other embodiments, the tissue site may be an unwanted fistula. As used herein, a “fistula” may broadly refer to an abnormal passage that leads from an abscess, hollow organ, or part to the body surface or from one hollow organ or part to another. The geometry and fluids involved may make treatment of fistulas difficult as well.

A surface wound, as used herein, is a wound on the surface of a body that is exposed to the outer surface of the body, such as an injury or damage to the epidermis, dermis, and/or subcutaneous layers. Surface wounds may include ulcers or closed incisions, for example. A surface wound, as used herein, does not include wounds within an intra-abdominal cavity. A wound may include chronic, acute, traumatic, subacute, and dehisced wounds, partial-thickness burns, ulcers (such as diabetic, pressure, or venous insufficiency ulcers), flaps, and grafts, for example.

The therapy system 100 may include a source or supply of negative pressure, such as a negative-pressure source 102, a dressing 104, a fluid container, such as a container 106, and a regulator or controller, such as a controller 108, for example. Additionally, the therapy system 100 may include sensors to measure operating parameters and provide feedback signals to the controller 108 indicative of the operating parameters. As illustrated in FIG. 1, for example, the therapy system 100 may include a first sensor 110, a second sensor 112, or both, coupled to the controller 108. As illustrated in the example of FIG. 1, the dressing 104 may comprise or consist essentially of one or more dressing layers, such as a tissue interface 114, a cover 116, or both in some embodiments.

The therapy system 100 may also include a source of instillation solution, such as saline, for example. For example, a solution source 118 may be fluidly coupled to the dressing 104, as illustrated in the example embodiment of FIG. 1. The solution source 118 may be fluidly coupled to a positive-pressure source such as the positive-pressure source 120, a negative-pressure source such as the negative-pressure source 102, or both in some embodiments. A regulator, such as an instillation regulator 122, may also be fluidly coupled to the solution source 118 and the dressing 104 to ensure proper dosage of instillation solution to a tissue site. For example, the instillation regulator 122 may comprise a piston that can be pneumatically actuated by the negative-pressure source 102 to draw instillation solution from the solution source during a negative-pressure interval and to instill the solution to a dressing during a venting interval. Additionally or alternatively, the controller 108 may be coupled to the negative-pressure source 102, the positive-pressure source 120, or both, to control dosage of instillation solution to a tissue site. In some embodiments, the instillation regulator 122 may also be fluidly coupled to the negative-pressure source 102 through the dressing 104, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the solution source 118, the controller 108 and other components into a therapy unit.

In general, components of the therapy system 100 may be coupled directly or indirectly. For example, the negative-pressure source 102 may be directly coupled to the container 106, and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to the controller 108. The negative-pressure source maybe fluidly coupled to one or more distribution components, which provide a fluid path to a tissue site. In some embodiments, components may also be coupled by virtue of physical proximity, being integral to a single structure, or being formed from the same piece of material. For example, the tissue interface 114 and the cover 116 may be discrete layers disposed adjacent to each other, and may be joined together in some embodiments.

A distribution component is preferably detachable, and may be disposable, reusable, or recyclable. The dressing 104 and the container 106 are illustrative of distribution components. A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components, including sensors and data communication devices. In some embodiments, for example, a dressing interface may facilitate coupling a fluid conductor to the dressing 104. For example, such a dressing interface may be a SENSAT.R.A.C.™ Pad available from kinetic Concepts, Inc. of San Antonio, Tex.

A negative-pressure supply, such as the negative-pressure source 102, may be a reservoir of air at a negative pressure, or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−9.9 kPa) and −300 mm Hg (−39.9 kPa).

The container 106 is representative of a container, canister, pouch, or other storage component, which can be used to manage exudates and other fluids withdrawn from a tissue site. In many environments, a rigid container may be used for collecting, storing, and disposing of fluids. In other environments, fluids may be properly disposed of without rigid container storage, and a re-usable container could reduce waste and costs associated with negative-pressure therapy.

A controller, such as the controller 108, may be a microprocessor or computer programmed to operate one or more components of the therapy system 100, such as the negative-pressure source 102. In some embodiments, for example, the controller 108 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 114, for example. In some embodiments, the controller 108 is particularly configured to receive one or more input signals, such as a feedback signal, and programmed to modify one or more operating parameters based on the input signals.

Sensors, such as the first sensor 110 and the second sensor 112, are generally known in the art as any apparatus operable to detect or measure a physical phenomenon or property, and generally provide a signal indicative of the phenomenon or property that is detected or measured. For example, the first sensor 110 and the second sensor 112 may be configured to measure one or more operating parameters of the therapy system 100. In some embodiments, the first sensor 110 may be a transducer configured to measure pressure in a pneumatic pathway and convert the measurement to a signal indicative of the pressure measured. In some embodiments, for example, the first sensor 110 may be a piezo-resistive strain gauge. The second sensor 112 may optionally measure operating parameters of the negative-pressure source 102, such as the voltage or current, in some embodiments. Particularly, the signals from the first sensor 110 and the second sensor 112 are suitable as an input signal to the controller 108, but some signal conditioning may be appropriate in some embodiments. For example, the signal may need to be filtered or amplified before it can be processed by the controller 108. Typically, the signal is an electrical signal, but may be represented in other forms, such as an optical signal.

The tissue interface 114 may be adapted to or configured to contact a tissue site. The tissue interface 114 may be partially or fully in contact with the tissue site. If the tissue site is a wound, for example, the tissue interface 114 may partially or completely fill the wound, or may be placed over the wound. The tissue interface 114 may take many forms and have more than one layer in some embodiments. The tissue interface 114 may also have many sizes, shapes, or thicknesses depending on a variety of factors, such as the type of treatment being implemented or the nature and size of a tissue site. For example, the size and shape of the tissue interface 114 may be adapted to the contours of deep and irregular shaped tissue sites.

In some embodiments, the cover 116 may provide a bacterial barrier and protection from physical trauma. The cover 116 may also be constructed from a material that can reduce evaporative losses and provide a fluid seal between two components or two environments, such as between a therapeutic environment and a local external environment. The cover 116 may comprise or consist of, for example, an elastomeric film or membrane that can provide a seal adequate to maintain a negative pressure at a tissue site for a given negative-pressure source. The cover 116 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 116 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 116 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 116 to an attachment surface, such as undamaged epidermis, a gasket, or another cover. The attachment device may take many forms. For example, an attachment device may be a medically-acceptable, pressure-sensitive adhesive configured to bond the cover 116 to epidermis around a tissue site, such as a tunnel wound or a fistula. In some embodiments, for example, some or all of the cover 116 may be coated with an adhesive, such as an acrylic adhesive, which may have a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 118 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

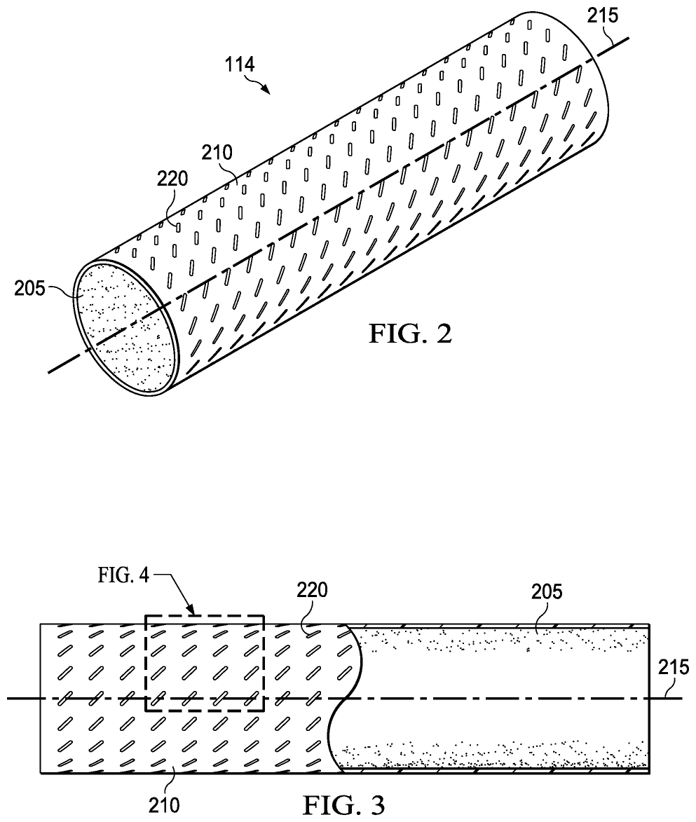

FIG. 2 is a perspective view of an example configuration of a tissue interface that may be associated with some example embodiments of the therapy system of FIG. 1. The tissue interface 114 may comprise a first layer 205 and a second layer 210. In some embodiments, the second layer 210 encloses or wraps around the first layer 205 completely or partially. For example, the second layer 210 may form an envelope or a sleeve around the first layer 205. The second layer 210 may be positioned in a circumferential orientation around the first layer 205. In some embodiments, the first layer 205 is bonded to the second layer 210. In some alternative embodiments, the first layer 205 is not bonded to the second layer 210. In some embodiments, the first layer 205 may not contact the tissue site or may not substantially contact the tissue site. For example, the second layer 210 may be configured to prevent or reduce tissue incorporation into the first layer 205.

In some embodiments, the tissue interface 114 may comprise or consist essentially of a tubular structure. For example, the first layer 205 may have a tubular shape with a central axis 215. As illustrated in the example of FIG. 2, the first layer 205 may have a circular cross-section. In other embodiments, the first layer 205 may have a cross-section that is semi-circular, elliptical, a polygonal, square, or hexagonal, for example. In some embodiments, the first layer 205 has a solid core. In other embodiments, the first layer 205 has a hollow core. The first layer 205 may be in the form of a cylinder or semi-cylinder in some embodiments. In some embodiments, the tissue interface 114, including the first layer 205, may accommodate or be configured to be adjacent to a tissue site.

In some embodiments, the first layer 205 has a solid body. In other embodiments, the first layer 205 has a hollow body. In some further embodiments, the first layer 205 has a first surface, a hollow body, and a second surface opposite the first surface. The thickness of the first layer 205 between the first surface and the second surface may vary according to needs of a prescribed therapy. The thickness of the first layer 205 can also affect the conformability of the first layer 205. In some embodiments, the first layer 205, such as a solid core with a circular cross-section, may have a diameter in a range of about 20 millimeters to 30 millimeters.

The first layer 205 may comprise or consist essentially of a manifold. A manifold may be used for the communication of pressure and the flow of fluids, such as wound fluids, instilled therapeutic fluid, air, or a combination thereof. In some embodiments, the manifold may be a hydrophilic, felted foam. In other embodiments, the manifold may be a foam that is not felted, such as an ether foam.

In further embodiments, the first layer 205 may provide a means for collecting or distributing fluid across the tissue interface 114 under pressure, or may be configured to collect or distribute fluid across the tissue interface 114 under pressure. For example, the first layer 205 may be configured to receive negative pressure from a source and distribute negative pressure through multiple apertures across the tissue interface 114, which may have the effect of collecting fluid from across a tissue site and drawing the fluid toward the source. In some embodiments, the fluid path may be reversed or a secondary fluid path may be provided to facilitate delivering fluid, such as from a source of instillation solution, across the tissue interface 114.

In some illustrative embodiments, the pathways of the first layer 205 may be interconnected to improve distribution or collection of fluids. In some illustrative embodiments, the first layer 205 may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the first layer 205 may comprise or consist essentially of a reticulated foam having pore sizes and free volume that may vary according to needs of a prescribed therapy. For example, a reticulated foam having a free volume of at least 90% may be suitable for many therapy applications, and a foam having an average pore size in a range of 400-600 microns may be particularly suitable for some types of therapy. The tensile strength of the first layer 205 may also vary according to needs of a prescribed therapy. For example, the tensile strength of a foam may be increased for instillation of topical treatment solutions. The 25% compression load deflection of the first layer 205 may be at least 0.35 pounds per square inch, and the 65% compression load deflection may be at least 0.43 pounds per square inch. In some embodiments, the tensile strength of the first layer 205 may be at least 10 pounds per square inch. The first layer 205 may have a tear strength of at least 2.5 pounds per inch. In some embodiments, the first layer 205 may be a foam comprised of polyols such as polyester or polyether, isocyanate such as toluene diisocyanate, and polymerization modifiers such as amines and tin compounds. In some non-limiting examples, the first layer 205 may be a reticulated polyurethane foam such as used in GRANUFOAM™ dressing or V.A.C. VERAFLO™ dressing, both available from Kinetic Concepts, Inc. of San Antonio, Tex.

In alternative and additional embodiments, the first layer 205 may comprise one or more perforations, such as a longitudinal perforation along the central axis 215 of the first layer 205. The perforation may have a diameter of between about 2 and about 5 millimeters. The perforation may help to manifold fluids through the first layer 205 and provide increased manifolding of fluids both to and from a tissue site. The first layer 205 may be configured to be coupled to a fluid delivery system and may also have within it a perforated polymer structure to assist with channeling fluids over the length of the structure.

In some embodiments, the second layer 210 may comprise or consist essentially of a means for controlling or managing fluid flow. The second layer 210 may comprise or consist essentially of a layer of a flexible polymer film, such as polyurethane, polyethylene, silicone, or any suitable flexible, conformable, and bio-compatible film. In some embodiments, the second layer 210 may comprise or consist essentially of a liquid-impermeable, elastomeric material. The second layer 210 may also have a smooth or matte surface texture in some embodiments. A glossy or shiny finish better or equal to a grade B3 according to the SPI (Society of the Plastics Industry) standards may be particularly advantageous for some applications.

In some embodiments, the second layer 210 may be hydrophobic. In particular embodiments, the second layer 210 is highly hydrophobic to reduce or prevent collection of biofilm and other materials on its surface. The hydrophobicity of the second layer 210 may vary, but may have a contact angle with water of at least ninety degrees in some embodiments. In some embodiments the second layer 210 may have a contact angle with water of no more than 150 degrees. For example, in some embodiments, the contact angle of the second layer 210 may be in a range of at least 90 degrees to about 120 degrees, or in a range of at least 120 degrees to 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things. Non-limiting examples of such integrated systems may include the FTÅ 125, FTÅ 200, FTÅ 2000, and FTÅ 4000 systems, all commercially available from First Ten Angstroms, Inc., of Portsmouth, Va., and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles described herein represent averages of 5-9 measured values, discarding both the highest and lowest measured values. The hydrophobicity of the second layer 210 may be further enhanced with a hydrophobic coating of other materials, such as silicones and fluorocarbons, either as coated from a liquid, or plasma coated.

The second layer 210 may also be suitable for welding to other layers, including the first layer 205. For example, the second layer 210 may be adapted for welding to polyurethane foams using heat, radio frequency (RF) welding, or other methods to generate heat such as ultrasonic welding. RF welding may be particularly suitable for more polar materials, such as polyurethane, polyamides, polyesters and acrylates. Sacrificial polar interfaces may be used to facilitate RF welding of less polar film materials, such as polyethylene.

The area density of the second layer 210 may vary according to a prescribed therapy or application. In some embodiments, an area density of less than 40 grams per square meter may be suitable, and an area density of about 20-30 grams per square meter may be particularly advantageous for some applications.

In some embodiments, for example, the second layer 210 may comprise or consist essentially of a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, polyether block polyamide copolymer (PEBAX) block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between 20 microns and 100 microns may be suitable for many applications. Films may be clear, colored, or printed. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations.

The second layer 210 may have one or more fluid restrictions 220, which can be distributed uniformly or randomly across the second layer 210. In some embodiments, the fluid restrictions 220 may be perforated through the partial or the whole length of the second layer 210. The fluid restrictions 220 may be bi-directional and pressure-responsive. For example, each of the fluid restrictions 220 may comprise or consist essentially of an elastic passage that can substantially reduce liquid flow if unstrained, and can expand or open in response to a pressure gradient. In some embodiments, the fluid restrictions 220 may comprise or consist essentially of perforations in the second layer 210. Perforations may be formed by removing material from the second layer 210. For example, perforations may be formed by cutting through the second layer 210, which may also deform the edges of the perforations in some embodiments. In the absence of a pressure gradient across the perforations, the passages may be sufficiently small to substantially reduce or prevent liquid flow.

Additionally or alternatively, one or more of the fluid restrictions 220 may be an elastomeric valve that is normally closed when unstrained to substantially prevent liquid flow, and can open or expand in response to a pressure gradient. The fluid restrictions 220 may comprise or consist essentially of fenestrations in the second layer 210. A fenestration in the second layer 210 may be a suitable valve for some applications. Fenestrations may also be formed by removing material from the second layer 210, but the amount of material removed and the resulting dimensions of the fenestrations may be up to an order of magnitude less than perforations, and may not deform the edges. Fenestrations may also be formed with no or insubstantial material removed from the second layer 210, such as through a laser cut.

The second layer 210 may wrap around and be bonded to the first layer 205, may be sprayed to the first layer 205, may be formed by a secondary heat-sealing process, or may be flame laminated to the first layer 205, for example. In further embodiments, the first layer 205 or the second layer 210 may be bonded or otherwise secured to one another with a solvent or non-solvent adhesive, or with thermal welding, for example, without adversely affecting fluid management. In particular embodiments, the second layer 210 may be bonded to the first layer 205 by a suitable acrylic adhesive, polyurethane adhesive or any other suitable adhesives. In other embodiments, the second layer 210 may be welded to form a tube, and radio frequency (RF) welding may be used to weld the second layer 210 to a section of the first layer 205.

One or more of the components of the dressing 104 may additionally be treated with an antimicrobial agent in some embodiments. For example, the first layer 205 may be a foam, mesh, or non-woven coated with an antimicrobial agent such as silver. In some embodiments, the first layer 205 may comprise antimicrobial elements, such as fibers coated with an antimicrobial agent such as silver. Additionally or alternatively, some embodiments of the second layer 210 may be a polymer coated or mixed with an antimicrobial agent such as silver. Suitable antimicrobial agents may include, for example, metallic silver, polyhexamethylene biguanide (PHMB), iodine or its complexes and mixes such as povidone iodine, copper metal compounds, chlorhexidine, or some combination of these materials.

The first layer 205 and the second layer 210 may be assembled before application. The first layer 205 may be encased in the second layer 210 in some embodiments. For example, the second layer 210 may be perforated and then wrapped around the first layer 205. An adhesive can attach the second layer 210 to the first layer 205 in some embodiments, which can provide a mechanical lock between the first layer 205 and the second layer 210 upon encirclement. In some embodiments, the process of manufacture may comprise, or consist essentially of, feeding the second layer 210 into a Delta Machine or other suitable automated assembly machine. The second layer 210 may then be perforated serially and then wrapped around the first layer 205, which may also be fed through the machine in sections. Additionally or alternatively, the first layer 205 and the second layer 210 may then be welded using RF or ultrasonics to seal the ends. In other embodiments, no adhesives are used for the assembly, and the second layer 210 may be rolled and opposing edges welded to form a tube. In further embodiments, the first layer 205 may be placed within the second layer 210 and welded to form a seal.

In use, the tissue interface 114 may be placed in or on a tissue site, and the cover 116 may be sealed to an attachment surface, such as epidermis peripheral to a tissue site, over the first layer 205 and the second layer 210. The geometry and dimensions of the tissue interface 114, the cover 116, or both may vary to suit a particular application or anatomy. For example, the geometry or dimensions of the tissue interface 114 and the cover 116 may be adapted to provide an effective and reliable seal at or around a tissue site. Additionally or alternatively, the dimensions may be modified to increase the surface area for the second layer 210 to enhance the movement and proliferation of epithelial cells at or around a tissue site and reduce the likelihood of granulation tissue in-growth.

Thus, the dressing 104 in the example of FIG. 2 can provide a sealed therapeutic environment proximate to a tissue site, substantially isolated from the external environment, and the negative-pressure source 102 can reduce the pressure in the sealed therapeutic environment. Negative pressure in the sealed environment can induce macrostrain and micro-strain in the tissue site in some embodiments. Negative pressure applied through the tissue interface 114 can also create a negative pressure differential across the fluid restrictions 220 in the second layer 210, which can open the fluid restrictions 220 to allow exudate and other liquid movement through the fluid restrictions 220. For example, in some embodiments in which the fluid restrictions 220 may comprise perforations through the second layer 210, a pressure gradient across the perforations can strain the adjacent material of the second layer 210 and increase the dimensions of the perforations to allow liquid movement through them, similar to the operation of a duckbill valve.

In some embodiments, the first layer 205 may be hydrophobic to minimize retention or storage of liquid in the dressing 104. In other embodiments, the first layer 205 may be hydrophilic. In an example in which the first layer 205 may be hydrophilic, the first layer 205 may also wick fluid away from a tissue site, while continuing to distribute negative pressure to the tissue site. The wicking properties of the first layer 205 may draw fluid away from a tissue site by capillary flow or other wicking mechanisms, for example. An example of a hydrophilic material that may be suitable for the first layer 205 is a polyvinyl alcohol, open-cell foam such as V.A.C. WHITEFOAM™ dressing available from KCI of San Antonio, Tex. Other hydrophilic foams may include those made from polyether. Other foams that may exhibit hydrophilic characteristics include hydrophobic foams that have been treated or coated to provide hydrophilicity.

If the negative-pressure source 102 is removed or turned-off, the pressure differential across the fluid restrictions 220 can dissipate, allowing the fluid restrictions 220 to return to an unstrained or resting state and prevent or reduce the return rate of exudate or other liquid moving to the tissue site through the second layer 210.

Additionally or alternatively, instillation solution or other fluid may be distributed to the dressing 104, which can increase the pressure in the tissue interface 114. The increased pressure in the tissue interface 114 can create a positive pressure differential across the fluid restrictions 220 in the second layer 210, which can open or expand the fluid restrictions 220 from their resting state to allow the instillation solution or other fluid to be distributed to the tissue site.

FIG. 3 is a partial cut-away view of the tissue interface of FIG. 2, illustrating additional details that may be associated with some example embodiments of the therapy system of FIG. 1. The fluid restrictions 220 of the second layer 210 may be configured to expose the first layer 205 to allow for effective manifolding to and from a tissue site. In some embodiments, the fluid restrictions 220 of the second layer 210 may be configured to avoid incorporation or granulation of tissue into the first layer 205.

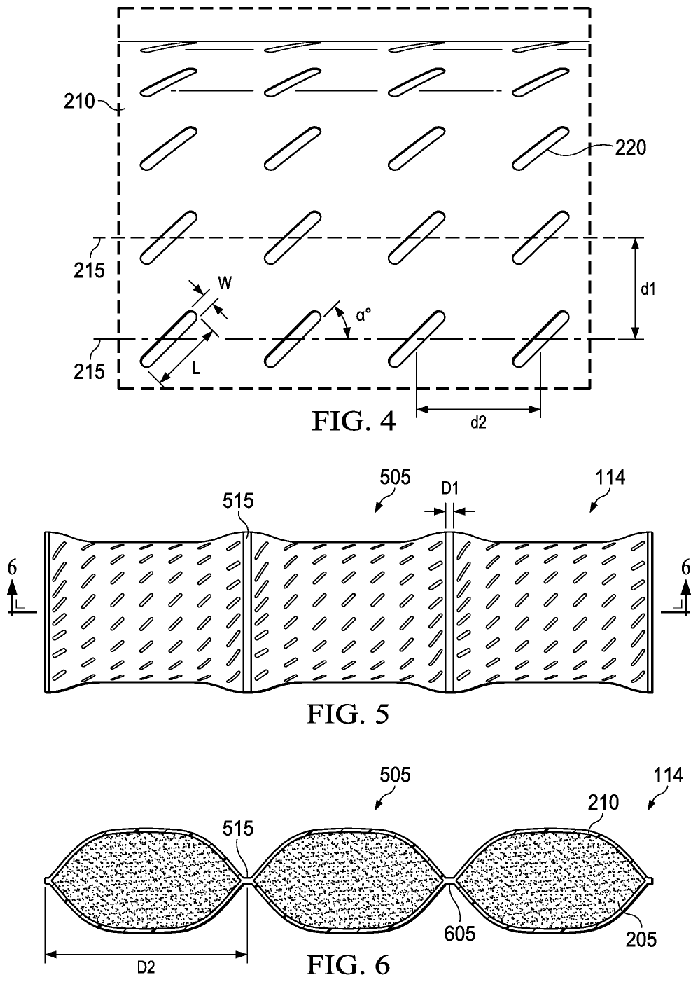

FIG. 4 is a partial detailed view of the tissue interface of FIG. 3. As illustrated in FIG. 4, the fluid restrictions 220 may have a line of symmetry that forms an angle α with the central axis 215 of the first layer 205. For example, the angle α may be an oblique angle in a range of about 30 degrees to about 60 degrees, particularly, about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 degrees, between about 35 degrees to about 55 degrees, between about 40 degrees to about 50 degrees. In a particular example, the oblique angle α is about 45 degrees. In other examples, an angle of 0 or 90 degrees may be suitable.

In some examples, the fluid restrictions 220 may comprise or consist essentially of linear slots or linear slits. In some embodiments, an example of a uniform distribution pattern of the fluid restrictions 220 may be provided. The fluid restrictions 220 may be substantially coextensive with the second layer 210, and may be distributed across the second layer 210 in a grid of parallel rows and columns, in which the slots are also mutually parallel to each other. In some embodiments, the rows may be spaced at a distance d1 of about 3 millimeters on center, and the fluid restrictions 220 within each of the rows may be spaced at a distance d2 of about 3 millimeters on center as illustrated in the example of FIG. 4. The fluid restrictions 220 in adjacent rows may be aligned or offset. For example, adjacent rows may be offset, so that the fluid restrictions 220 are aligned in alternating rows and separated by about 6 millimeters. The spacing of the fluid restrictions 220 may vary in some embodiments to increase the density of the fluid restrictions 220 according to therapeutic requirements.

As illustrated in the example of FIG. 4, the fluid restrictions 220 may have a length L of less than 4 millimeters and a width W of less than 1 millimeter. The length L may be at least 2 millimeters, and the width W may be at least 0.4 millimeters in some embodiments. In particular embodiments, the fluid restrictions 220 may have a length L of about 3 millimeters. A length L of about 3 millimeters and a width W of about 0.8 millimeters may be particularly suitable for many applications, and a tolerance of about 0.1 millimeter may also be acceptable. Such dimensions and tolerances may be achieved with a laser cutter, for example. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow.

FIG. 5 is a top view of another example configuration of a tissue interface, illustrating additional details that may be associated with some embodiments. As illustrated in FIG. 5, the tissue interface 114 may be separated into a plurality of separable sections. In the example of FIG. 5, the tissue interface 114 comprises one or more interface sections 505, which may be bounded by one or more seams 515. The seams 515 may have a width D1 of about 5 to about 8 mm in some embodiments. The example configuration of FIG. 5 may be used in combination with or instead of other configurations of the tissue interface 114.

FIG. 6 is a section view of the tissue interface of FIG. 5, illustrating additional details that may be associated with some examples. In the example of FIG. 6, the seams 515 may be formed by one or more bonds 605 between opposing portions of the second layer 210. The bonds 605 may be continuous or discrete. For example, the bonds 605 may be formed by welding the second layer 210 at a set distance D2 along a length of the tissue interface 114. In some embodiments, a distance D2 of about 3 cm to about 5 cm may be suitable. In some examples, the bonds 605 may be formed by welding the second layer 210 through the first layer 205. Additionally or alternatively, the second layer 210 may be bonded through perforations (not shown) in the first layer 205. The seams 515 preferably reduce or eliminate exposure of the first layer 205. In some embodiments, the seams 515 may allow the communication of fluid and pressure and may not form a complete pneumatic or fluid seal. In the example of FIG. 6, the interface sections 505 may be separated to size the tissue interface 114 without exposing the first layer 205 to a tissue site.

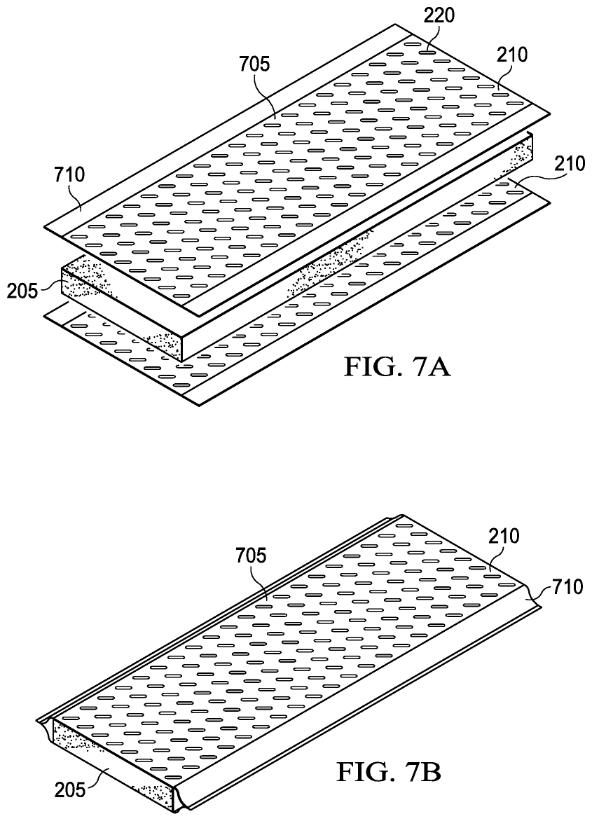

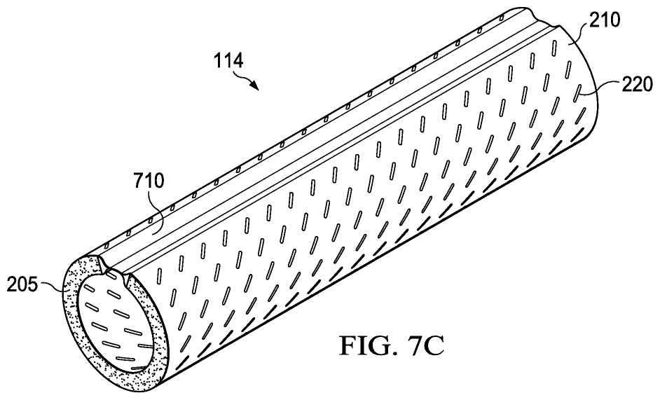

FIGS. 7A-7C illustrate another example configuration of a tissue interface in various stages of assembly. In the example of FIGS. 7A-7C, the first layer 205 has a rectangular cross-section, and the second layer 210 may be formed with two films 705. In some embodiments, the first layer 205 may have a thickness of about 5 to about 8 millimeters.

As illustrated in FIG. 7A, the two films 705 may be applied to opposing sides of the first layer 205. As illustrated in FIG. 7B, edges 710 of the films 705 may be welded to form the second layer 210, which may be a sleeve or an envelope around the first layer 205.

As illustrated in FIG. 7C, the first layer 205 and the two films 705 may be rolled lengthwise, and the edges 710 of the second layers 210 may be joined together to form a tube or hollow cylinder. The second layer 210 (with fluid restrictions 220) may be disposed adjacent to both inner and outer surfaces of the first layer 205 form a tissue interface 114. The example configuration prepared according to FIGS. 7A-7C may be used in combination with or instead of other configurations of the first layer 205 and the second layer 210 described above.

The systems, apparatuses, and methods described herein may provide significant advantages. For example, some embodiments of the dressing 104 may provide a long-application deep-wound filler structure for use in or around complex wounds with a longer change frequency. In some embodiments, the dressing 104 may be able to place the instillation or cleanse solution in and around deep and tunneled wounds or around structures such as implanted metalwork and fixation to clean and reduce infection, such as osteomyelitis in bones. Additionally, some embodiments of the dressing 104 may be implanted around infected bone, limbs, or tissues with an extended duration time without the need for further surgical debridement upon removal. In particular embodiments, the dressing 104 may be left in place for at least five days, six days, seven days, eight days, nine days or ten days, or up to seven to ten days. Some embodiments of the dressing 104 may be combined with negative-pressure therapy to clean and reduce infection in a deep wound or around structures such as implanted metalwork and fixation.

Additionally, it has been found, unexpectedly, that the fluid restrictions 220 having an oblique angle of between about 30 degrees to about 60 degrees, particularly, about 45 degrees, can be favorable for the delivery of pressure and the removal of fluids.

In some embodiments, the improved duration of the dressing 104 can improve the clinical outcome by reducing disturbance to the patient and wound. The dressing 104 may also provide a significant cost saving for the healthcare system by reducing the skill and facilities required for each wound intervention and dressing change or removal.

Some embodiments of the dressing 104 may remain on the tissue site for at least 5 days, and some embodiments may remain for at least 7 days. Antimicrobial agents in the dressing 104 may extend the usable life of the dressing 104 by reducing or eliminating infection risks that may be associated with extended use, particularly use with infected or highly exuding wounds. In some embodiments, the tissue interface 114 can substantially reduce or prevent in-growth of tissue from a tissue site.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context.

Features, elements, and aspects described in the context of some embodiments may also be omitted, combined, or replaced by alternative features serving the same, equivalent, or similar purpose without departing from the scope of the invention defined by the appended claims. For example, one or more of the features of some layers may be combined with features of other layers to provide an equivalent function.

Components may be also be combined or eliminated in various configurations for purposes of sale, manufacture, assembly, or use. For example, in some configurations the dressing 104, the container 106, or both may be eliminated or separated from other components for manufacture or sale. In other example configurations, components of the dressing 104 may also be manufactured, configured, assembled, or sold independently or as a kit.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for treating a tissue site with negative pressure, the dressing comprising:
a manifold having a tubular shape with a central axis;
a polymer film substantially enclosing the manifold; and
a plurality of fluid restrictions in the polymer film, the fluid restrictions configured to expand in response to a pressure gradient across the polymer film.

2. The dressing of claim 1, wherein the polymer film is hydrophobic.

3. (canceled)

4. (canceled)

5. (canceled)

6. (canceled)

7. The dressing of claim 1, wherein the fluid restrictions comprise a plurality of slots configured to permit fluid flow and inhibit exposure of the manifold to the tissue site.

8. The dressing of claim 7, wherein each of the plurality of slots has a line of symmetry that forms an oblique angle with the central axis of the manifold.

9. The dressing of claim 8, wherein the oblique angle is in a range of about 30 degrees to about 60 degrees.

10. The dressing of claim 8, wherein the oblique angle is about 45 degrees.

11. The dressing of claim 1, wherein the fluid restrictions comprise a plurality of slots, each of the slots having a length of at least 2 millimeters and not greater than 4 millimeters,

12. The dressing of claim 1, wherein the fluid restrictions comprise a plurality of slots, each of the slots having a width of at least 0.5 millimeters and not greater than 2 millimeters.

13. The dressing of claim 1, wherein the fluid restrictions comprise a plurality of slots, each of the slots having a length of at least 2 millimeters and not greater than 4 millimeters, and having a width of at least 0.5 millimeters and not greater than 2 millimeters.

14. The dressing of claim 1, wherein the fluid restrictions comprise or consist essentially of elastomeric valves in the polymer film that are normally closed.

15. The dressing of claim 14, wherein the elastomeric valves are fenestrations.

16. The dressing of claim 14, wherein the elastor xeric valves are slits.

17. The dressing of claim 1, wherein the fluid restrictions comprise a plurality of slits in the polymer film.

18. The dressing of claim 17, wherein the plurality of slits comprise linear slits that form an oblique angle with the central axis of the manifold.

19. The dressing of claim 18, wherein the oblique angle is in a range of about 30 degrees to about 60 degrees.

20. The dressing of claim 18, wherein the oblique angle is about 45 degrees.

21. The dressing of claim 17, wherein each of the plurality of slits has a length of at least 2 millimeters and not greater than 4 millimeters.

22. (canceled)

23. The dressing of claim 1, wherein the manifold is hydrophilic,

24. The dressing of claim 1, wherein the manifold comprises foam.

25. The dressing of claim 24, wherein the foam is a polymer foam.

26. (canceled)

27. The dressing of claim 24, wherein the foam is open-cell foam.

28. (canceled)

29. The dressing of claim 24, wherein the foam is a reticulated polymer foam.

30. (canceled)

31. The dressing of claim 24, wherein the foam is felted.

32. The dressing of claim 1, wherein the tubular shape has an elliptical cross-section.

33. The dressing of claim 1, wherein the tubular shape has a circular cross-section.

34. The dressing of claim 1, wherein the tubular shape has a semi-circular cross-section.

35. The dressing of claim 1, wherein the tubular shape has a polygon cross-section.

36. The dressing of claim 1, wherein the tubular shape has a hollow core.

37. The dressing of claim 1, further comprising a plurality of bonds between portions of the polymer film, the plurality of bonds defining separable sections of the manifold.

38. The dressing of claim 37, wherein the plurality of bonds form seams between the separable sections of the manifold.

39. The dressing of claim 37, wherein the plurality of bonds form seams at intervals of about 3 centimeters to about 5 centimeters.

40. The dressing of claim 38, wherein the seams have a length in a range of about 5 millimeters to about 8 millimeters.

41. The dressing of claim 37, further comprising perforations through the manifold aligned with the bonds.

42. The dressing of claim 38, wherein the manifold comprises perforations between the separable sections.

43. A dressing for treating a tissue site with negative pressure, the dressing comprising:
a manifold comprising a first surface and a second surface opposite the first surface;
an envelope around the manifold, the envelope defined by a first polymer film and a second polymer film coupled to a periphery of the first polymer film; and
a plurality of fluid restrictions in the envelope, the fluid restrictions configured to expand in response to a pressure gradient across the polymer film;
wherein the manifold is formed into a tubular shape, and the envelope comprises a first edge and a second edge coupled to the first edge to retain the tubular shape.

44. (canceled)
